(19) United States
(12) Patent Application Publication
(10) Pub. No.: US 2025/0029687 A1
(43) Pub. Date: Jan. 23, 2025

(54) SYSTEM AND METHOD FOR PREDICTION OF THE LIKELIHOOD OF THE RESPONSE TO PLACEBO DURING CLINICAL TRIALS FROM RAW SCALP EEG AND ACCOMPANIED METADATA

(71) Applicant: Neuroscience Software Inc. DBA Brianify.AI, Dover, DE (US)

(72) Inventors: Ivan Mishanin, New York City, NY (US); Mariam Khayretdinova, Dover, DE (US); Alexey Shovkun, Dover, DE (US); Andrey Kirysov, Dover, DE (US); Ilya Zakharov, Dover, DE (US); Maxim Popov, Dover, DE (US); Andrey Zhdanov, Dover, DE (US); Polina Pshonkovskaya, Dover, DE (US)

(21) Appl. No.: 18/777,325

(22) Filed: Jul. 18, 2024

Related U.S. Application Data
(60) Provisional application No. 63/514,220, filed on Jul. 18, 2023.

(51) Int. Cl. G16H 10/20; A61B 5/00; A61B 5/291; A61B 5/372; G16H 10/60

(52) U.S. Cl. CPC G16H 10/20; A61B 5/291; A61B 5/372; A61B 5/7203; A61B 5/7225; A61B 5/725; A61B 5/7267; G16H 10/60

(57) ABSTRACT

A system and method for prediction of the likelihood of the response to placebo during clinical trials from raw scalp EEG and accompanied metadata. To accomplish this, the system includes an electroencephalography (EEG) device, at least one EEG data, at least one measured metadata, at least one machine learning unit, and at least one segmented EEG data. The method of use may include the steps of collecting behaviorally measured metadata, collecting EEG data, uploading EEG data, segmenting EEG data, performing data augmentation and channel rolling on the EEG data, performing a model inference, performing a prediction aggregation, outputting a result, and transmitting a result to an end user or client. Predicting the likelihood of a placebo response of a subject in advance assists in tailoring the subjects of the clinical trial. This will save time and money and improve the resulting data from the clinical trial.

### FIELD OF THE INVENTION

The present invention relates generally to statistical prediction systems and methods. Specifically, the present invention is a system and method for predicting the likelihood of response to placebo during clinical trials, represented as an individual score derived from raw scalp electroencephalography (EEG) and accompanied metadata.

### BACKGROUND OF THE INVENTION

The gold standard for testing any kind of intervention is the clinical trial that is randomized, double-blind and placebo controlled. Such trials typically include two arms: one for treatment and one for control (placebo). A placebo is a “sham” intervention that is designed to have no treatment effect. The goal is to observe a statistically significant difference in treatment effect between the two groups and demonstrate treatment's superiority over placebo. However, patients that are subjected to placebos in clinical studies often do show responses in what is known as the placebo effect. The placebo effect is one of the main sources of high variance seen in subjects' responses in depression studies. High variance becomes the key hurdle towards demonstrating a statistically significant difference between the treatment group and the control (placebo) group. The effect is particularly noticeable in depression studies, with it being one of the main causes for the significant number of failures in the development of novel antidepressant treatments. Specifically, high placebo-response rates reduce the rates of positive outcomes in phase II and III clinical trials. In order to speed up the process of phase II clinical trials and increase the statistical power of such studies, it is proposed that trial volunteers who are likely to respond to a placebo be excluded from such studies at the screening stage, and this decision is likely to yield results that are beneficial to the researchers and pharmaceutical companies involved.

Overall, studies have shown that the most general predictors of placebo response included psychological constructs related to actions, expected outcomes, and the emotional valence attached to these events (goal-seeking, self-efficacy/-esteem, locus of control, optimism, Horing et al., 2014). Among other predictors involved behavioral control (desire for control, eating restraint), personality variables (fun-seeking, sensation seeking, neuroticism), biological markers (sex, a single nucleotide polymorphism related to dopamine metabolism), suggestibility and beliefs in expectation biases, body consciousness, and baseline symptom severity were found to be predictive. The role of the expectancy factors was especially high for placebo response in pain-related drug testing (Vase et al., 2015). For various types of drugs, one of the important factors was also the baseline level of the symptoms to be reduced and the age of the participants (Ballou et al., 2018; Shinohara et al., 2019, Trivedi et al., 2018).

The brain characteristics as the base for predictions were tested in pain and depression studies. Resting-state fMRI-derived brain connectivity was shown to be predictive of placebo response in two pain clinical trials (Tetreault et al., 2016). The individual characteristics of placebo non-responders, specifically for depression patients and EEG signals, were identified in studies by Diego Pizzagalli, on a group level (Trivedi et al., 2018; Ang et al., 2022). Some of these characteristics were related to the general state of the patient (e.g., a younger age, high cognitive processing speeds, or an absence of a history of physical abuse). Other characteristics were those hypothesized to be related to specific subtypes of depression (as characterized by factors such as levels of anxiety or anhedonia). Independently, it was shown that the probability that an individual will respond to a placebo could be predicted from their EEG resting state (also supported by a recent meta-analysis by Whatts et al., 2022). The predictive EEG characteristics include both the more basic quantitative features of the signal (e.g., higher rostral theta current density) and the more sophisticated EEG functional connectivity measures (i.e., greater alpha-band and lower gamma-band connectivity-most prominently parietal).

With placebo response factoring prominently into receiving accurate clinical trial data, there thus exists a need for an efficient method to screen clinical trial participants for placebo responses in advance of a clinical trial. To this end, the present invention is a system and method for prediction of the likelihood of the response to placebo during clinical trials from raw scalp EEG and accompanied metadata.

### SUMMARY OF THE INVENTION

The present invention is a system and method for prediction of the likelihood of the response to placebo during clinical trials from raw scalp EEG and accompanied metadata. To that end, the present invention may comprise an electroencephalography device (hereinafter, “EEG device”), at least one EEG data, at least one measured metadata, at least one machine learning unit, and at least one segmented EEG data. The method of use may comprise the steps of collecting behaviorally measured metadata, collecting electroencephalography (hereinafter “EEG”) data, uploading EEG data, segmenting EEG data, performing data augmentation and channel rolling on the EEG data, performing a model inference, performing a prediction aggregation, outputting a result, and transmitting a result to an end user or client. The goal of the present invention is to predict the likelihood of a placebo response of a subject for a clinical trial in advance of a clinical trial to identify placebo non-responders in advance to assist in tailoring the subjects of the clinical trial. This will save time and money and improve the resulting data from the clinical trial.

To that end, both behavioral and EEG data are used. First, a potential candidate (hereinafter “candidate”) for a clinical trial is identified. The candidate is then asked a series of questions or subjected to tests to acquire behavioral metadata. Next, the candidate is given an EEG test using an EEG device. The EEG data may comprise a two-minute eye open task, a two-minute eye closed task, or other similar EEG data collection tasks well-known in the art. Once EEG data has been collected, the EEG data may be uploaded server-side for data preprocessing.

During data preprocessing, unnecessary artifacts are removed, and eye-related activity is deleted. Once the data has been processed and cleared of artifacts, the data is segmented in an EEG segmentation step for use with the model inference. Ideally, the EEG data is segmented into shorter segments that are devoid of artifacts, each segment being roughly a few seconds long, though other lengths of the segments are contemplated. Once the data has been segmented, the data is augmented in a data augmentation step.

The data augmentation step performs functions such as gaussian noise application, random amplification, shrinking or stretching of the time axis, and inverse time flow of the EEG channels. A channel rolling method extends the receptive field of a first convolutional layer to all EEG channels, providing more thorough results.

Once augmented, the data is passed to a model inference step. The model inference step uses a deep learning model for accurate placebo response prediction. The predicted probability of an EEG segment to belong to a placebo-responder is output by the deep learning model. The model output is a real number in a range between zero and one. This process is then repeated in the prediction aggregation step. The deep learning model predicts a probability for each EEG segment received, and then aggregates these probabilities based on the number of segments to output a numerical probability of placebo response in the individual based on the totality of the data received.

In some embodiments, the above may be implemented using a Software as a Service (SaaS) model, wherein cloud solutions may be used to assist in data transfer, and an API is used to expose the resulting placebo response output to third party systems. In other embodiments, the above may be implemented as a local software run entirely on a local machine, foregoing the need of any cloud computing or network connection. A training solution may be used for training and improving the run-time model, which may comprise both training data and a development model including architecture, learning strategy, loss and activation functions and other (hyper)parameters required to develop a predictive model. Client third party systems for diagnosis and treatment may use the REST API of the production cloud solution to obtain the predicted placebo response score. Incoming EEG recordings may be preprocessed and further analyzed by the deep convolutional neural network on the service side by production cloud solutions. As more data is processed, the deep learning model will continue to learn and improve, and improvements shown by the training model may be deployed to the deep learning model in the production environment in real time.

### BRIEF DESCRIPTIONS OF THE DRAWINGS

FIG. 1 is a system diagram of the present invention.

FIG. 2 is a flowchart of the overall method of the present invention in detail.

FIG. 3 shows a flowchart of the method of the present invention.

FIG. 4 shows a flowchart of the method of the present invention.

FIG. 5 shows a flowchart of the method of the present invention.

FIG. 6 shows a flowchart of the method of the present invention.

FIG. 7 shows a flowchart of the method of the present invention.

FIG. 8 shows a flowchart of the method of the present invention.

FIG. 9 is an exemplary embodiment of a Software as a Service implementation of the present invention.

### DETAIL DESCRIPTIONS OF THE INVENTION

All illustrations of the drawings are for the purpose of describing selected versions of the present invention and are not intended to limit the scope of the present invention. Unless otherwise stated and in addition to what is stated, it should be presumed in the following writings that any step that involves data processing may be performed on any device, machine, or server that comprises a processing device such as a central processing unit or similar device well-known in the art. Unless otherwise stated and in addition to what is stated, it should be presumed that any device or step that involves storing data may store the data on a hard disk, hard drive, solid state drive, database, or any other computer readable medium well-known in the art. Unless otherwise stated and in addition to what is stated, it should be presumed that any step or device involving data transfer may use any means of data transfer well-known in the art, such as transfer of data via IP packets, direct data transfer using a hardwire, local area network data transfer using local Wi-Fi, and should be presumed to use standard security and encryption measures that are well-known in the art.

The present invention provides a system and a method for predicting response of individuals to placebo during clinical trials.

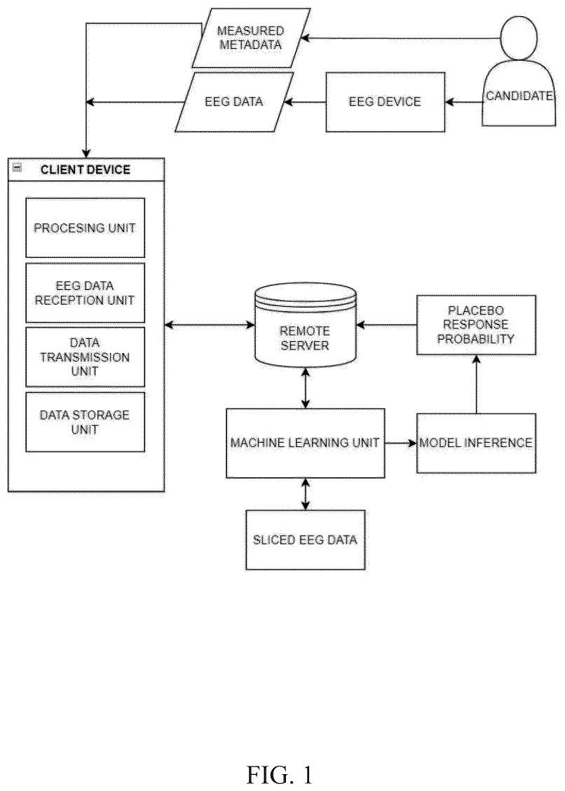

The following description is in reference to FIG. 1 through FIG. 9.

According to a preferred embodiment, a system for executing an overall method of the present invention comprises providing a client device managed by at least one remote server (Step A). The client device may comprise any computing device well-known in the art. As seen in FIG. 1, the client device may comprise a processing unit, an EEG data reception unit, a data transmission unit, and a data storage unit. The processing unit may comprise any data processor well-known in the art for performing computation and calculations. The EEG data reception unit may comprise any means well-known in the art for connecting to the EEG device, such as a USB connection, local wireless data connection, or other means for data transfer with an EEG device well-known in the art. The data transmission unit may comprise any means of data transfer over the internet or other network that is well-known in the art, such as a wi-fi connection, ethernet connection, or similar means for data transfer over a network. The data storage unit may comprise a hard drive, solid state drive, USB stick, or other data storage medium that is well-known in the art and is adapted to store EEG data prior to transmission.

The remote server may comprise a cloud server, database, or other remote data storage medium well-known in the art.

The system for executing the overall method further comprises providing at least one machine learning unit managed by the at least one remote server (Step B). The at least one machine learning unit may comprise a processing unit or server adapted to perform machine learning algorithms. Further, the overall method of the present invention comprises providing an electroencephalography (EEG) device, wherein the EEG device is adapted to acquire scalp EEG data of a candidate (Step C). The EEG device may comprise any EEG apparatus well-known in the art. The EEG apparatus is ideally adapted with a plurality of electrodes, the plurality of electrodes being adapted both to acquire scalp EEG data and for attachment near the eyes of the candidate. In the ideal embodiment, the electrodes may be adapted to be placed in the following configuration: 3 mm above the left eyebrow, 1.5 cm below the left bottom eye lid, and 1.5 cm lateral to the outer canthus of each eye of the candidate. It should be understood that other configurations or placements of the electrodes are within the spirit and scope of the present invention.

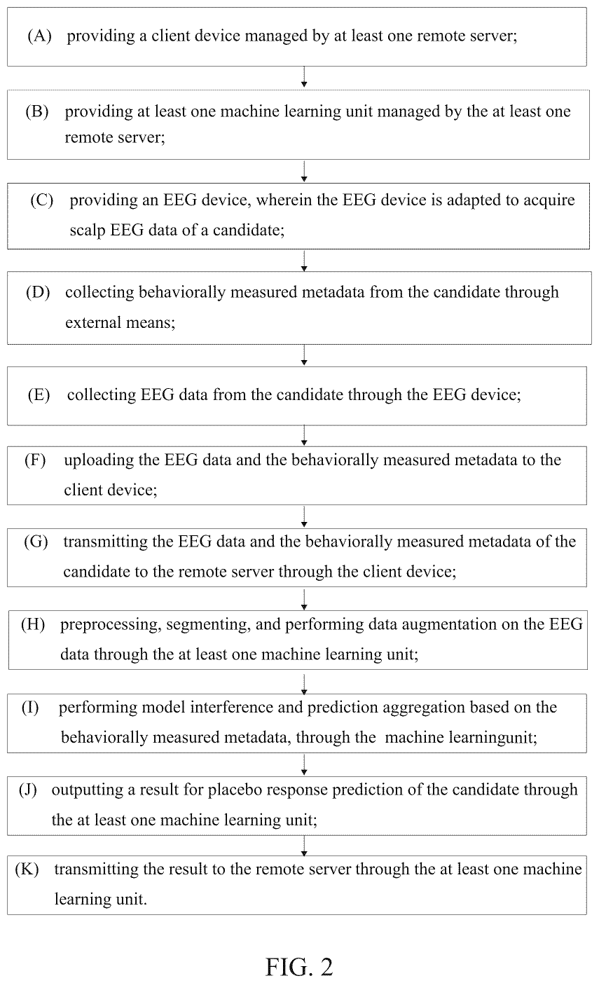

As seen in FIG. 2, The overall method of the present invention comprises collecting behaviorally measured metadata from the candidate through external means (Step D). The behaviorally measured metadata may comprise data obtained from a potential candidate for a clinical trial. The at least one behaviorally measured metadata may comprise demographic data, behavioral data, or other similar data obtained via questionnaire or testing of the candidate.

Continuing with the preferred embodiment, the overall method continues by collecting EEG data from the candidate through the EEG device (Step E). The at least one EEG data recording may comprise a data set created by running the EEG device after the electrodes have been attached to the candidate. In the ideal embodiment, the at least one EEG data recording may comprise a 2-minute eye open task and a 2-minute eye closed task. Other tests and EEG data types that are well-known in the art are within the spirit and scope of the present invention. Subsequently, the overall method continues with the steps of uploading the EEG data and the behaviorally measured metadata to the client device (Step F) and transmitting the EEG data and the behaviorally measured metadata of the candidate to the remote server through the client device (Step G). In other words, the method further comprises the step of uploading data to the remote server. The data to be uploaded may comprise both the EEG data, behaviorally measured data, any client-side instructions for the remote server or processing, or any other data that may be relevant for calculating the placebo response score. The data may be either saved locally to the client device, then uploaded, or may be uploaded directly during and after testing to the remote server. The data may be uploaded fully or in segments.

In some embodiments, the data may undergo data preprocessing. The data preprocessing step may be performed by any capable processing device, but is ideally performed by the machine learning unit, and may be performed using a machine learning algorithm. Accordingly, the overall method continues by preprocessing, segmenting, and performing data augmentation on the EEG data through the at least one machine learning unit (Step H). The data preprocessing acts to remove unnecessary artifacts from the EEG data. The method further comprises the step of segmenting the EEG data. The EEG data is ideally split into several second long EEG segments by a processing device, and in some embodiments, the segments may be split using the machine learning unit. In the ideal embodiment, the segments of EEG data may be roughly four seconds in length, though other lengths are contemplated. In some embodiments, only the artifact-free segments produced during the data preprocessing are used, in which case the length of viable segments may be limited. Testing has found the optimal EEG segment length to be roughly four seconds. The method further comprises performing data augmentation and channel rolling on the EEG data. This step is ideally performed by the machine learning unit using a deep convolutional neural network.

The overall method further continues by performing model interference and prediction aggregation based on the subject's EEG and behaviorally measured metadata, through the at least one machine learning unit (Step I). More specifically, the model inference step involves using the machine learning unit to provide an accurate placebo response prediction for the candidate. Further, the overall method continues by outputting a result for placebo response prediction of the candidate through the at least one machine learning unit (Step J) and transmitting the result to the remote server through the at least one machine learning unit (Step K). In other words, a result may be output and stored in the machine learning unit or database, and the method further comprises transmitting the result to an end user or client. The results may be transmitted over the network to the end user using any data transmission means that is well-known in the art.

A more detailed description of the present invention follows.

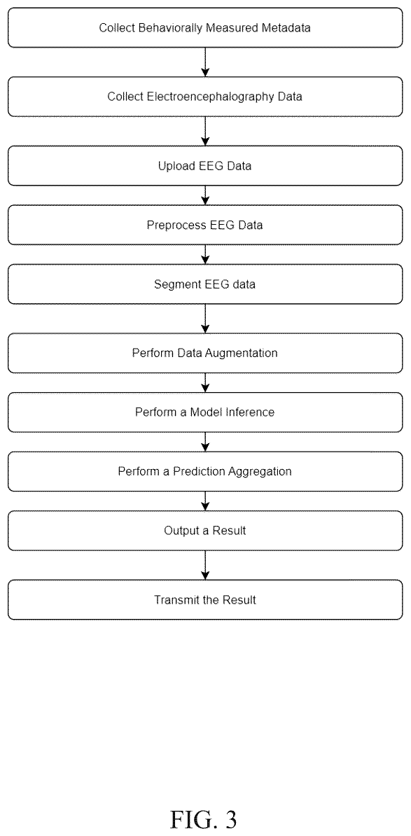

As shown in FIG. 3 through FIG. 9, the method of use may comprise the steps of collecting behaviorally measured metadata, collecting electroencephalography data, uploading EEG data, segmenting EEG data, performing data augmentation and channel rolling on the EEG data, performing a model inference, performing a prediction aggregation, outputting a result, and transmitting a result to an end user or client.

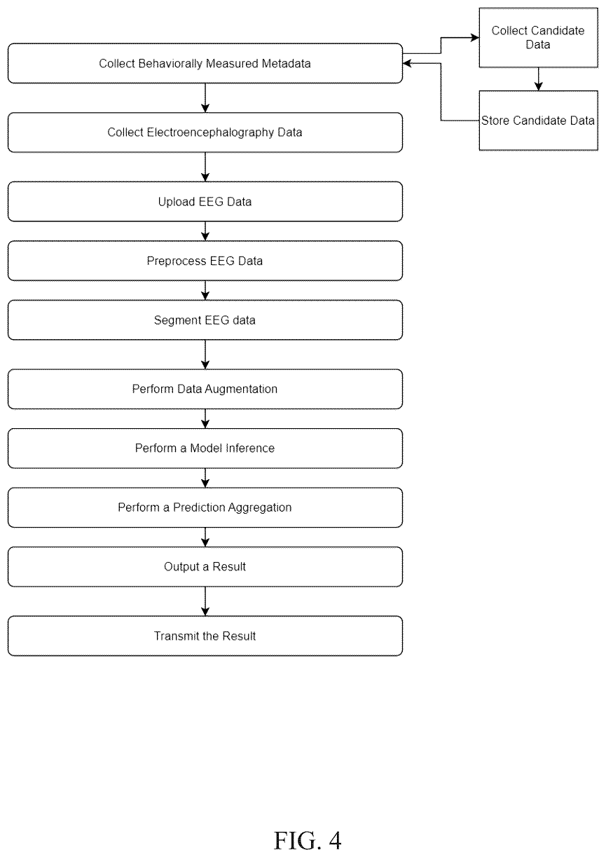

In reference to FIG. 4, the first step of the method comprises the steps of collecting behaviorally measured metadata. Further, external means of collecting behaviorally measured metadata comprise at least one of digital means and physical means. In other words, the behaviorally measured metadata may be collected from the candidate using either digital means, such as an online survey, or physical means, such as physical testing or a locally administered on-site questionnaire. The collected behaviorally measured metadata may include demographic data, behavioral data, or other similar data obtained via questionnaire or testing of the candidate that may be relevant to the placebo response prediction.

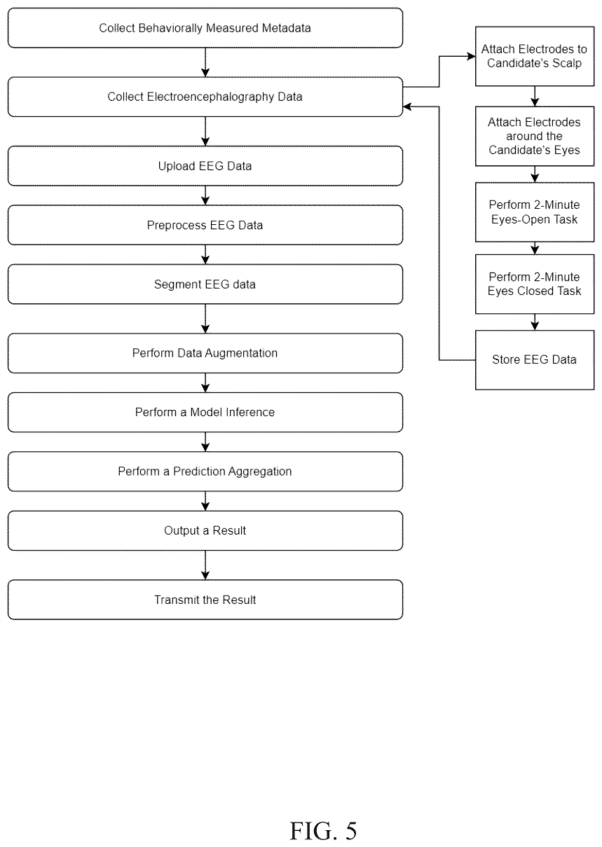

In reference to FIG. 5, the method comprises the step of collecting EEG data. According to the preferred method, collecting EEG data from the candidate comprises a sub-process with the steps of attaching electrodes to the candidate's scalp, attaching electrodes around the candidate's eyes, taking EEG measurements using resting state eyes closed condition for the candidate, taking EEG measurements using resting state eyes open conditions for the candidate, and storing the EEG data in the client device. In other words, in the ideal embodiment, the step of collecting EEG data may comprise attaching electrodes to the candidate, measuring EEG data using the EEG device and saving the EEG data obtained from the candidate. In the ideal embodiment, the electrodes may be placed in the following configuration, in addition to electrodes placed in accordance with standard EEG practice on the candidate's scalp: 3 mm above the left eyebrow, 1.5 cm below the left bottom eye lid, and 1.5 cm lateral to the outer canthus of each eye of the candidate. It should be understood that other configurations or placements of the electrodes are within the spirit and scope of the present invention. Once the electrodes are attached, the EEG data collected from the candidate may comprise a two-minute eye open task, a two-minute eye closed task, or other similar EEG data collection tasks well-known in the art.

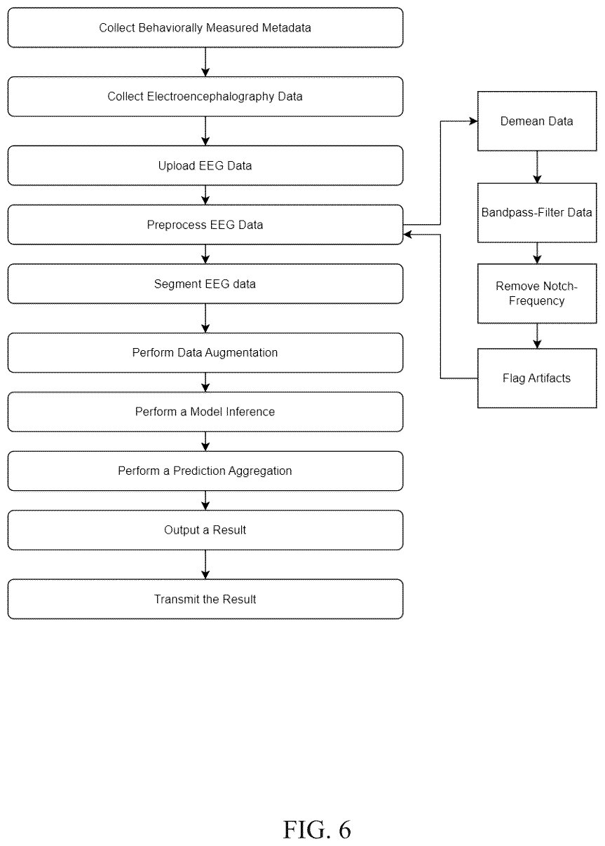

In reference to FIG. 6, preprocessing the EEG data further comprises a sub-process, with the steps of demeaning the EEG data, bandpass-filtering the EEG data, removing notch-frequencies from the EEG data, and flagging artifacts from the EEG data. More specifically, this is ideally accomplished using the previously known art of automatic preprocessing routines (Dijk et al, 2022, Jas et al., 2017). The EEG data is demeaned and bandpass-filtered between 0.5 and 100 hz, and the notch-frequency of 50 Hz or 60 Hz is removed from the EEG data. An extra artifacts channel may be added to the data with an artifact flag. The artifact flag may be used to indicate whether various artifacts signals are detected or not detected based on a detection algorithm. The detection algorithm may detect: electromyography (EMG) activity, sharp channel-jumps (up and down, kurtosis, extreme voltage swing, residual eye blinks, electrode bridging (Alschuler et al., 2014), and extreme correlations. Eye-related activity is deleted either with method proposed by Gratton et. al., 1983 or based on ICA decomposition of the signal.

In some embodiments, the EEG data may be sliced into the at least one segmented EEG data recording prior to processing by the machine learning unit. In the ideal embodiment, each slice of the at least one segmented EEG data recording may be roughly four seconds in length, though other lengths are contemplated. The data may be sliced based on a variety of input factors. Some input factors may include slicing the data to be a certain length, slicing the data to remove artifacts, or other similar input factors to tailor the data to user requirements.

In the preferred embodiment, the at least one machine learning unit may comprise a convolutional neural network. The machine learning unit may learn discriminative time and spatial features by being trained on raw EEG recordings. In some embodiments, the machine learning unit may use a single-layer perceptron to solve the classification task. As explained further herein, the machine learning unit may be pre-trained, may be able to be trained while deployed, and may perform data augmentation and channel rolling to improve results. In the ideal embodiment, the at least one machine learning unit is adapted to receive EEG recordings from a candidate, slice the EEG recordings into smaller segments, augment the EEG recordings to remove noise and artifacts, and perform a model inference based on the EEG data and the at least one measured metadata observation to output a placebo response probability for the candidate.

Turning now to the convolutional neural network of the machine learning unit, Convolutional Neural networks (CNNs) have shown great success in different pattern recognition and computer vision applications. This is due to the ability of CNN to automatically extract significant spatial features that best represent the data from its raw form without any preprocessing and without any human decision in selecting these features. The sparse connectivity and parameter sharing of CNN give it high superiority regarding the memory footprint as it requires much less memory to store the sparse weights. The equivariant representation property of the CNN increases the detection accuracy of a pattern when it exists in a different location across the input signal. A typical CNN comprises three types of layers: convolution layer, pooling layer, and fully connected layer. The convolution layer is used to generate the feature map by applying filters with trainable weights to the input data. This feature map is then down-sampled by applying the pooling layer to reduce the features' dimension and, therefore, the computational complexity. Finally, the fully connected layer is applied to all the preceding layer's output to generate the one-dimensional feature vector. CNN is used as a feature extractor to replace the complex feature engineering used in the prior art. In some embodiments of the present invention, the CNN may receive an EEG segment that has been converted into a 3D matrix to be suitable for use with a deep CNN. The CNN may receive as input the output of a channel rolling operation, described in greater detail further in this document. The middle part of the deep CNN architecture model may comprise four convolutional blocks. Each convolution block may contain a convolution operation followed by a batch normalization and activation function. Table 1, below, shows an exemplary embodiment for the parameters of operation for each convolutional block.

$${Conv}_{ch , t} = {\sum }_{m = - ∞}^{∞} {\sum }_{n = - ∞}^{∞} {\sum }_{c = 1}^{{N}_{c}} W [ c , m , n ] * I [ c , ch - m , t - n ] ,$$

where c is an input tensor channel, ch is an EEG channel, t is a time-point.

TABLE 1
Convolution blocks of the deep CNN claimed.

| | Convolution parameters | | | | |
|---|---|---|---|---|---|
| Convolution | | Kernel | | Batch | Activation |
| Block | Kernels | size | Stride | normalization | function |
| conv-1 | 16 | (7, 64) | (1, 3) | Yes | SiLU |
| conv-2 | 32 | (7, 32) | | | (aka “swish-1”) |
| conv-3 | 64 | (7, 16) | | | |
| conv-4 | 128 | (7, 8) | | | |

The batch Normalization technique is used to improve the training convergence and reduce overfitting. Both the input and output of a Batch Normalization layer are four-dimensional tensors, which refers to as I_{b,c,ch,t} and BN_{b,c,ch,t}, where b corresponds to examples within a mini-batch. Batch Normalization applies the same normalization for all activations in a given channel,

$${BN}_{b , c , ch , t} = {γ}_{c} \frac{{I}_{b , c , ch , t} - {μ}_{c}}{\sqrt{{σ}_{c}^{2} + ε}} + {β}_{c} , ∀ b , c , ch , t . \qquad ( 6 )$$

Here, Batch Normalization subtracts the mean activation

$${μ}_{c} = \frac{1}{\vert B \vert} {\sum }_{b , ch , t}$$

I_{b,c,ch,t} from all input activations in channel c, where B contains all activations in channel c across all features b in the entire mini-batch and all “spatial” (ch, t) locations. Subsequently, Batch Normalization divides the centered activation by the standard deviation c (plus for numerical stability) which is calculated analogously. During testing, running averages of the mean and variances are used. Normalization is followed by a channel-wise affine transformation parametrized through c, c, which are learned during training.

A Sigmoid-weighted Linear Unit (SiLU) activation function, as defined by (7), is used across the convolution layers to add nonlinearity, ensure robustness against noise in the input data, and achieve faster backpropagation convergence.

$$silu ( x ) = x * σ ( x ) \qquad ( 7 )$$

$$σ ( x ) = \frac{1}{1 + {e}^{- x}} \qquad ( 8 )$$

The final part of deep CNN consists of a Global Average Pooling (9) layer and a single Linear (10) layer, also referenced as a Single-Layer Perceptron, of size 128 and Sigmoid activation function (8), which are responsible for the placebo response probability prediction.

$${GlobalAVGPoolI}_{b , c} = \frac{1}{{N}_{ch} * {N}_{t}} {\sum }_{m = 0}^{{N}_{ch}} {\sum }_{n = 0}^{{N}_{t}} {I}_{b , c , m , n} , \qquad ( 9 )$$

where N_{ch} and Nₜ are sizes of input tensor across EEG channel and time dimensions, respectively.

$${Linear}_{b} = W * {I}_{b} + {b}_{0} , \qquad ( 10 )$$

where W and b₀ are learned parameters.

The loss function used is the binary cross-entropy defined by (11)

$$bce ( y , \hat{y} ) = - ( y * log log ( \hat{y} ) + ( 1 - y ) * log log ( 1 - \hat{y} ) ) \qquad ( 11 )$$

where ŷ and yare predicted and target classes, respectively.

In the ideal embodiment, the machine learning unit may be trained with back propagation using an Adam optimization algorithm with the starting learning rate 3e-5, reduce-on-plateau scheduler with a patience of three epochs, and early stopping after ten epochs without validation metric improvement.

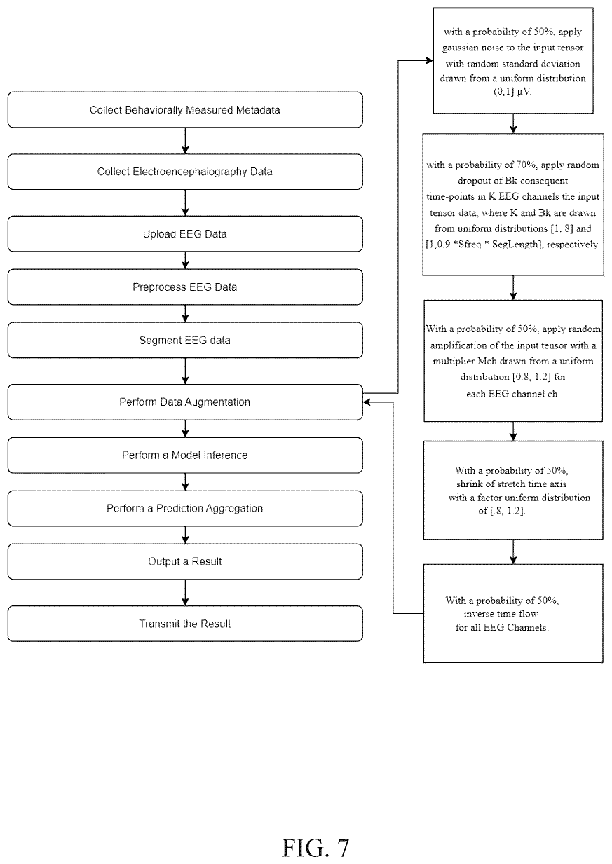

Thus, according to the preferred embodiment, the at least one machine learning unit employees a deep convolutional neural network for performing data augmentation and channel rolling on the EEG data. In reference to FIG. 7, the training method for the deep convolutional neural network may involve the use of a data augmentation method for each EEG segment. The data augmentation techniques may include, but are not limited to:
with a probability of 50%, apply gaussian noise to the input tensor with random standard deviation drawn from a uniform distribution (0,1] μV.
with a probability of 70%, apply random dropout of Bₖ consequent time-points in K EEG channels the input tensor data, where K and Bₖ are drawn from uniform distributions [1, 8] and [1, Len_{seg}*SFreq*0.9], respectively, where Len_{seg} is the length of one EEG segment in seconds and SFreq is the sampling frequency of raw EEG data.
with a probability of 50%, apply random amplification of the input tensor with a multiplier M_{ch} drawn from a uniform distribution [0.8, 1.2] for each EEG channel ch.
With a probability of 50%, shrink or stretch time axis with a factor uniform distribution [0.8, 1.2].
With a probability of 50%, inverse time flow for all EEG channels.

One of the main features of deep learning convolutional neural networks is the repeated use of the convolution operation: the convolution of the first layer is applied to the input data, the convolution of the 2nd layer to the output of the 1st layer, etc. The receptive field specifies which region of the input the convolution operation will process to get one point in the output.

The deep learning unit may ideally use a “channel rolling” method to extend the receptive field of the first convolutional layer to all N_{ch} EEG channels. For this, the input tensor is shifted by seven along the EEG channels dimension (dimension #1) 3 times in a turn. The resulting tensors are then stacked with the original tensor along dimension #0. After this operation, the original tensor with dimensions [1, N_{ch}, Sfreq*SegLength] turns into a tensor with dimensions [4, N_{ch}, Sfreq*SegLength]. Once the EEG data has been augmented, it may be ready to be passed into a model inference.

More specifically, the channel rolling method extends the receptive field of the first convolutional layer of the network to all input EEG channels and is defined by the following algorithm:
xᵢₙ−the input tensor with shape (xᵢₙ)=[1, N_{channels}, Len_{seg}*SFreq], where Len_{seg} is the length of one EEG segment in seconds and SFreq is the sampling frequency of raw EEG data.

$${N}_{steps} = ⌈ \frac{{N}_{channels}}{{KernelSize}_{channels}} ⌉ ,$$

the resulting number of channels in the output tensor dimension #0, where KernelSize_{channels} is the size of the kernel of the first convolutional layer for the EEG channels dimension.
xₒᵤₜ=xᵢₙ, xᵢ=xᵢₙ
for i in [2 . . . Nₛₜₑₚₛ]:
   i. xᵢ=roll(xᵢ, KernelSize, 1)−roll tensor xᵢ by KernelSize shifts along EEG channels dimension #1.
   ii. xₒᵤₜ=concatenate(xₒᵤₜ, xᵢ, 0)−concatenate tensors xₒᵤₜ and xᵢ in the dimension #0.
return xₒᵤₜ

Continuing with the preferred embodiment, for model training purposes the placebo response prediction is formulated as a binary classification task, where a result of a 0 indicates no placebo response, while a result of 1 indicates a placebo response. The model inference uses data from the behaviorally measured metadata, an EEG segment, and any additional information provided by the client. The result of the model inference is a real number from [0, 1] range which is the likelihood of an EEG segment belonging to a placebo-responder person. The model inference may use the deep learning convolution neural network or other machine learning algorithm to process the input data and output the placebo response prediction. This step is repeated for each EEG segment produced during the EEG segmentation and data preprocessing steps. Thus, in other words, the result of placebo response prediction is at least one of 0 and 1, wherein a result of 0 indicates no placebo response for the candidate, and a result of 1 indicates a placebo response for the candidate.

Some studies may argue that the placebo prediction problem is a continuous measure as it varies from person to person. However, the prediction problem may be reduced to a continuous placebo prediction task, which, in turn, is solved as a part of the binary classification task of a subject. In short, the deep model is trained as a binary classifier, then the raw predicted probability of placebo is used to calculate the resulting score.

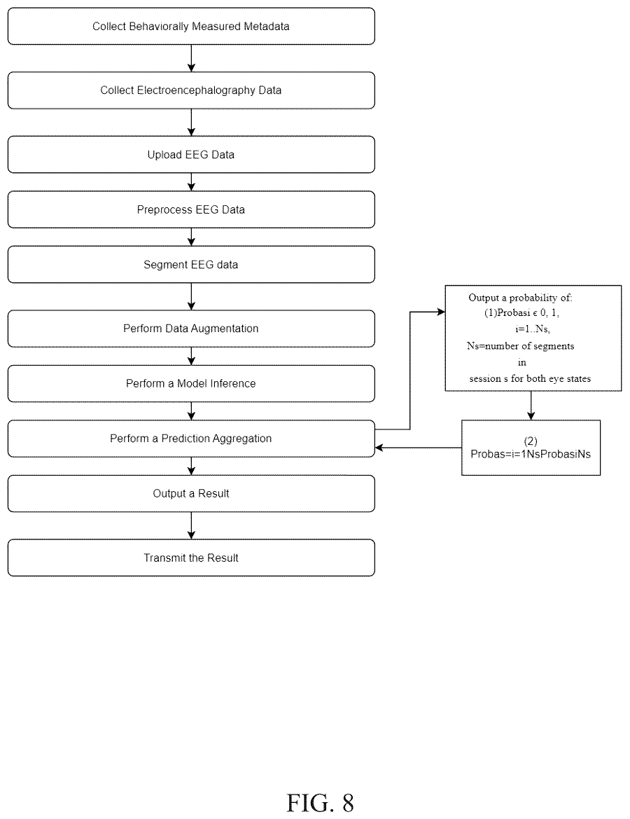

As seen in FIG. 8, the method further comprises performing a prediction aggregation. The prediction aggregation combines each placebo response prediction produced during the model inference step. The probabilities are averaged over EEG segments for both eye states to obtain a session-level placebo response probability. This probability can be expressed with the following equation:

$$\begin{matrix}{Proba}_{s}^{i} ϵ [ 0 , 1 ] , i = 1 … {N}_{s} , {N}_{s} \\ = number of segments in session s for both eye states\end{matrix} \qquad ( 1 )$$

$${Prob}_{s} = \frac{{\sum }_{i = 1}^{{N}_{s}} {Proba}_{s}^{i}}{{N}_{s}} \qquad ( 2 )$$

The method further comprises outputting a result. A result of the above equations (1) and (2) may be output and stored in the machine learning unit or database. In some embodiments, the result may be a number between 0 and 1 that represents the likelihood of placebo response. As noted prior, it should be understood that other formats or configurations of the result are contemplated.

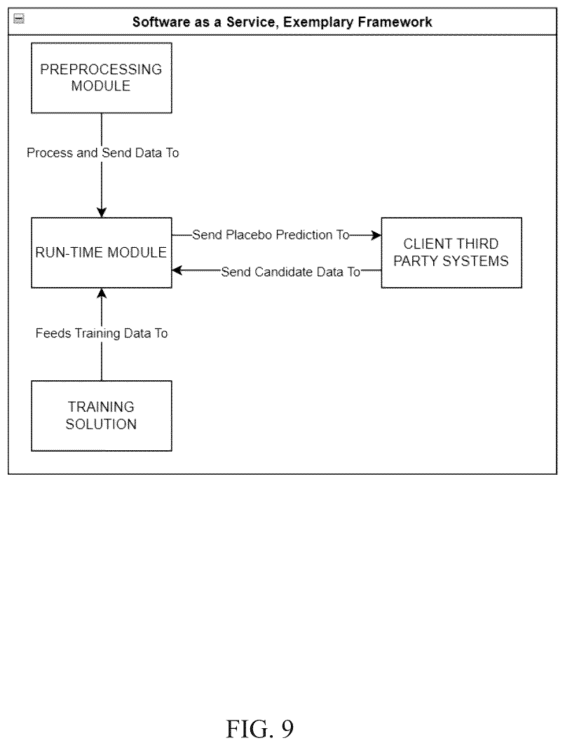

In reference to FIG. 9, in some embodiments, the method described above may be implemented as a Software as a Service (SaaS) framework. More specifically, a SaaS framework may be employed for performing step (H) through step (K), wherein an exemplary embodiment of the SaaS framework comprises a preprocessing module, a run-time module, a training solution, and client third party systems.

The preprocessing module may comprise any processor or network capable of performing the preprocessing step, above. Such processors and networks capable of data and algorithm processing are well-known in the art.

The run-time module may be responsible for the placebo response probability calculation and prediction logic. The run-time module may be any computing or process unit that is adapted to handle implementation of the machine learning model. In some embodiments, the run time model may simply comprise the at least one machine learning unit.

The training solution may be used for training and improving the run-time module. The training solution may comprise a development model, the development model comprises model architecture, a learning strategy, a plurality of loss and activation functions, and other similar (hyper)parameters required to develop the predictive model for placebo response. The training solution may further comprise or receive training data, such as sample EEG data for training.

Client third party systems may be used in conjunction with the preprocessing module, run-time module, and training solution. The client third party systems may be adapted for diagnosing and treating candidates or patients. The client third party systems may use a REST API of the SaaS framework to obtain the predicted placebo response score. The client third party system may be adapted to send candidate data to the SaaS framework, where the data will be preprocessed and analyzed by the at least one machine learning unit.

Although the invention has been explained in relation to its preferred embodiment, it is to be understood that many other possible modifications and variations can be made without departing from the spirit and scope of the invention.

What is claimed is:

1. A method of predicting response of individuals to placebo during clinical trials, the method comprising:
(A) providing a client device managed by at least one remote server;
(B) providing at least one machine learning unit managed by the at least one remote server;
(C) providing an EEG device, wherein the EEG device is adapted to acquire scalp EEG data of a candidate;
(D) collecting behaviorally measured metadata from the candidate through external means;
(E) collecting EEG data from the candidate through the EEG device;
(F) uploading the EEG data and the behaviorally measured metadata to the client device;
(G) transmitting the EEG data and the behaviorally measured metadata of the candidate to the remote server through the client device;
(H) preprocessing, segmenting, and performing data augmentation on the EEG data through the at least one machine learning unit;
(I) performing model interference and prediction aggregation based on the behaviorally measured metadata, through the at least one machine learning unit;
(J) outputting a result for placebo response prediction of the candidate through the at least one machine learning unit; and
(K) transmitting the result to the remote server through the at least one machine learning unit.

2. The method of claim 1, wherein external means of collecting behaviorally measured metadata comprises at least one of digital means and physical means.

3. The method of claim 1, wherein the client device comprises wherein the client device comprises a processing unit, an EEG data reception unit, a data transmission unit, and a data storage unit.

4. The method of claim 1, wherein collecting EEG data from the candidate further comprising:
attaching electrodes to the candidates scalp;
attaching electrodes around the candidates eyes;
taking EEG measurements using resting state eyes closed condition for the candidate;
taking EEG measurements using resting state eyes open conditions for the candidate; and
storing the EEG data in the client device.

5. The method of claim 1, wherein preprocessing the EEG data further comprising:
demeaning the EEG data;
bandpass-filtering the EEG data;
removing notch-frequencies from the EEG data; and
flagging artifacts from the EEG data.

6. The method of claim 1, wherein the at least one machine learning unit employees a deep convolutional neural network for performing data augmentation and channel rolling on the EEG data.

7. The method of claim 6, wherein data augmentation employs the following algorithm:
with a probability of 50%, apply gaussian noise to the input tensor with random standard deviation drawn from a uniform distribution (0,1] μV.
with a probability of 70%, apply random dropout of Bₖ consequent time-points in K EEG channels the input tensor data, where K and Bₖ are drawn from uniform distributions [1, 8] and [1, Len_{seg}*SFreq*0.9], respectively, where Len_{seg} is the length of one EEG segment in seconds and SFreq is the sampling frequency of raw EEG data.
with a probability of 50%, apply random amplification of the input tensor with a multiplier M_{ch} drawn from a uniform distribution [0.8, 1.2] for each EEG channel ch.
With a probability of 50%, shrink or stretch time axis with a factor uniform distribution [0.8, 1.2].
With a probability of 50%, inverse time flow for all EEG channels.

8. The method of claim 6, wherein channel rolling employs the following algorithm:
xᵢₙ−the input tensor with shape(xᵢₙ)=[1, N_{channels},Len_{seg}*SFreq], where Len_{seg} is the length of one EEG segment in seconds and SFreq is the sampling frequency of raw EEG data.
$${N}_{steps} = ⌈ \frac{{N}_{channels}}{{KernelSize}_{channels}} ⌉ ,$$
the resulting number of channels in the output tensor dimension #0, where KernelSize_{channels} is the size of the kernel of the first convolutional layer for the EEG channels dimension.
xₒᵤₜ=xᵢₙ, xᵢ=xᵢₙ
for i in [2 . . . Nₛₜₑₚₛ]:
xᵢ=roll(xᵢ, KernelSize, 1)−roll tensor xᵢ by KernelSize shifts along EEG channels dimension #1.
xₒᵤₜ=concatenate(xₒᵤₜ, xᵢ, 0)−concatenate tensors xₒᵤₜ and xᵢ in the dimension #0.
return xₒᵤₜ.

9. The method of claim 1, wherein the result of placebo response prediction is at least one of 0 and 1.

10. The method of claim 9, wherein:
a result of 0 indicates no placebo response for the candidate; and
a result of 1 indicates a placebo response for the candidate.

11. The method of claim 1, wherein a Software as a Service (SaaS) framework is employed for performing step (H) through step (K).

12. The method of claim 11, wherein the SaaS framework comprises a preprocessing module, a run-time module, a training solution, and client third party systems.

13. A method of predicting response of individuals to placebo during clinical trials, the method comprising:
(A) providing a client device managed by at least one remote server, wherein the client device comprises wherein the client device comprises a processing unit, an EEG data reception unit, a data transmission unit, and a data storage unit;
(B) providing at least one machine learning unit managed by the at least one remote server;
(C) providing an EEG device, wherein the EEG device is adapted to acquire scalp EEG data of a candidate;
(D) collecting behaviorally measured metadata from the candidate through external means, wherein external means comprises at least one of digital means and physical means;
(E) collecting EEG data from the candidate through the EEG device;
(F) uploading the EEG data and the behaviorally measured metadata to the client device;
(G) transmitting the EEG data and the behaviorally measured metadata of the candidate to the remote server through the client device;
(H) preprocessing, segmenting, and performing data augmentation on the EEG data through the at least one machine learning unit;
(I) performing model interference and prediction aggregation based on the behaviorally measured metadata, through the at least one machine learning unit;
(J) outputting a result for placebo response prediction of the candidate through the at least one machine learning unit; and transmitting the result to the remote server through the at least one machine learning unit.

14. The method of claim 13, wherein collecting EEG data from the candidate further comprising:
attaching electrodes to the candidates scalp;
attaching electrodes around the candidates eyes;
taking EEG measurements using resting state eyes closed condition for the candidate;
taking EEG measurements using resting state eyes open conditions for the candidate; and
storing the EEG data in the client device.

15. The method of claim 13, wherein preprocessing the EEG data further comprising:
demeaning the EEG data;
bandpass-filtering the EEG data;
removing notch-frequencies from the EEG data; and
flagging artifacts from the EEG data.

16. The method of claim 1, wherein the at least one machine learning unit employees a deep convolutional neural network for performing data augmentation and channel rolling on the EEG data.

17. The method of claim 16, wherein data augmentation employs the following algorithm:
with a probability of 50%, apply gaussian noise to the input tensor with random standard deviation drawn from a uniform distribution (0,1] μV.
with a probability of 70%, apply random dropout of Bₖ consequent time-points in K EEG channels the input tensor data, where K and Bₖ are drawn from uniform distributions [1, 8] and [1, Len_{seg}*SFreq*0.9], respectively, where Len_{seg} is the length of one EEG segment in seconds and SFreq is the sampling frequency of raw EEG data.
with a probability of 50%, apply random amplification of the input tensor with a multiplier M_{ch} drawn from a uniform distribution [0.8, 1.2] for each EEG channel ch.
With a probability of 50%, shrink or stretch time axis with a factor uniform distribution [0.8, 1.2].
With a probability of 50%, inverse time flow for all EEG channels.

18. The method of claim 16, wherein channel rolling employs the following algorithm:
xᵢₙ−the input tensor with shape (xᵢₙ)=[1, N_{channels}, Len_{seg}*SFreq], where Len_{seg} is the length of one EEG segment in seconds and SFreq is the sampling frequency of raw EEG data.
$${N}_{steps} = ⌈ \frac{{N}_{channels}}{{KernelSize}_{channels}} ⌉ ,$$
the resulting number of channels in the output tensor dimension #0, where KernelSize_{channels} is the size of the kernel of the first convolutional layer for the EEG channels dimension.
xₒᵤₜ=xᵢₙ, xᵢ=xᵢₙ
for i in [2 . . . Nₛₜₑₚₛ]:
xᵢ=roll(xᵢ, KernelSize, 1)−roll tensor xᵢ by KernelSize shifts along EEG channels dimension #1.
xₒᵤₜ=concatenate(xₒᵤₜ, xᵢ, 0)−concatenate tensors xₒᵤₜ and xᵢ in the dimension #0.
return xₒᵤₜ.

19. The method of claim 1, wherein the result of placebo response prediction is at least one of 0 and 1.

20. The method of claim 1, wherein a Software as a Service (SaaS) framework is employed for performing step (H) through step (K).
